(19) United States
(12) Patent Application Publication
(10) Pub. No.: US 2009/0221708 A1
(43) Pub. Date: Sep. 3, 2009

(54) Medicinal preparation

(76) Inventor: Mikhail Vladimirovich Kutushov, Moscow (RU)

Correspondence Address: PAULEY PETERSEN & ERICKSON, 2800 WEST HIGGINS ROAD, SUITE 365, HOFFMAN ESTATES, IL, 60169, US

(21) Appl. No.: 11/578,879

(22) PCT Filed: Mar. 31, 2005
(86) PCT No.: PCT/RU2005/000155

§ 371 Date: May 8, 2009

(30) Foreign Application Priority Data
Apr. 22, 2004 (RU) 2004112187

(51) Int. Cl. A61K 31/192; A61P 29/00; A61P 37/00

(52) U.S. Cl. 514/571

(57) ABSTRACT

Medicine, in particular broad spectrum medicinal preparations. This invention is based on the use of 4-chlorine-2-methyl-phenoxy-acetic acid of formula I, the pharmacologically acceptable derivatives thereof, for example on corresponding alkali metal salts of the acid and the derivatives in the form of pharmaceutical preparations exhibiting immunomodulating, anti-inflammatory and anti-tumoral properties and antiviral activity. The broad spectrum of therapeutic modalities of the inventive medicinal preparation and the fact that it does not produce side effects are proven by study.

### BACKGROUND OF THE INVENTION

1. Field of the Invention

This invention relates to a medicinal sector, more especially to medicaments having a wide effective spectrum.

2. Discussion of Related Art

The preparation Fluoruracil is known, which is a white or slightly yellowish, difficulty water-soluble and alcohol-soluble, crystalline powder and an antimetabolic agent. The antiblastomatous activity of this preparation is determined by its conversion, in cancer cells, into a competition inhibitor, which participates in the synthesis of a ferment of nucleic acids. See, for example, M. D. Maschkowskij, Drugs, Moscow, Nowaja Wolna/Neue Welle GmbH, publisher S.B. Diwow, 2002, Volume 2, P. 425.

The preparation is used as intravenous injections for inoperable tumours and recidive tumours of the stomach, cancers of the large intestine and rectum, cancers of the mammary gland and ovary, as well as cancer of the pancreas. However, the preparation has a high toxicity, and includes a possible suppression of blood formation, diarrhoea, appetite reduction, vomiting and ulcer-like stomatitis when used. Furthermore, a counter-indication is associated with the preparation in the case of a generally serious condition of the patient, in the case of stomach and duodenal ulcers and pronounced liver failure.

The nearest prototype is Reaferon, which represents a recombinant α₂ Interferon, which is produced from the pseudomonad bacterial strain. A gene of the leucocytic, human α₂ Interferon is incorporated in the gene system of α2 Interferon. The α₂ Interferon is produced as a porous powder, the aqueous solution of which is used as intramuscular and subcutaneous injections. See, for example, M. D. Maschkowskij, Drugs, Moscow, Nowaja Wolna/Neue Welle GmbH, publisher S.B. Diwow, 2002, Volume 2, P. 323-324.

The preparation has an immune-modulating, antiblastomatous and antiviral activity, works effectively in the treatment of viral hepatitis and is used, inter alia, in the treatment of hairy cell leukaemia or Kaposhi sarcomas (in the case of AIDS), renal cancers and metastatic melanomas. However, with its use, side effects are possible such as shaking, general feeling of being unwell, allergic skin reactions, leukocytopenia and thrombocytopenia.

### SUMMARY OF THE INVENTION

One object of this invention is to provide a drug which has a wide healing effect spectrum and causes no side effects with its use in pharmaceutically viable doses.

4-chlorine-2-methyl phenoxyacetic acid is known, which represents a colorless, water-soluble and alcohol-soluble, crystalline substance and is used, inter alia, in agronomy as a herbicide under the trade names 2M-4X, MCPA, Metoxon (see “Katalog Schadstoffe in der Industrie” [in translation; Catalogue of Pollutants in Industry], published by Verlag Chemie, Leningrad, 1976, P. 131).

Through tests, this acid was detected in the internal organs and in the blood of the people who were involved in the production and use of this acid, it being predominantly removed from the organism with the urine (see Bache C. A. et al, Daire Sci., 1964, Vol. 47, P 93-95).

According to recommendations of the Research Institute WNIIGINTOKS, for the production of this acid and its derivatives, a maximum permissible concentration of these substances is stipulated in production areas. For example, 1 mg/m³ is stipulated for sodium salt (see Manual for carrying out the hygiene monitoring of working conditions in the production of 2M-4X herbicide [Metoxon], Ufa, 1971).

### DETAILED DESCRIPTION OF THE INVENTION

Furthermore, it was ascertained, from the biochemical investigations of the herbicidal effect on plant growth factors, that the acid influences the energy transformation in the cell, increases the intensity of the oxidation processes and suppresses the phosphorylation in the mitochondria. In such case, a disturbance of the complex, adhering to the membranes of the protein factor, is caused by a herbicidal accumulation in the membranes. The activity of the RNA polymerase is determined, which synthesises mRNAs determined by a transcription. With a change in the functional activity of the membranes and the enzymes adhering thereto, herbicides with gamete properties can trigger an induction of the synthesis of mRNAs, which are responsible for the production of the enzymes and hence the ferments, which transform the carbohydrate components of the membranes and cell coverings (see Hardin J. W., Morre D. J., Lembi C. A., Enhancement of soybean R'NA polymerase activity by a factor released by auxin from plasma-membrane; Proc. Naitl. Acad. Sci., USA, 1972, Vol. 69, N 11, P. 31146-31150).

These data lead to the assumption that some herbicides have a similar effect on the human organism.

The object of this invention can be achieved by a drug which possesses immune-modulating, inflammation-inhibiting and antiblastomatous properties as well as an antiviral activity, and includes 4-chlorine-2-methyl phenoxyacetic acid with the structural formula:

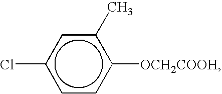

and/or contains its pharmacologically viable derivatives, for example salts of the alkali metals or mixtures of these salts or respectively mixtures of 4-chlorine-2-methyl phenoxyacetic acid and these derivatives.

In such case, the derivative of 4-chlorine-2-methyl phenoxyacetic acid represents either a potassium salt or a sodium salt or a lithium salt of this acid.

Furthermore, the MCPA derivative represents either a mixture of potassium salt and sodium salt of the acid in the proportion of 1.0-98.0% by wt. potassium salt and residual sodium salt, or a mixture of 1.0-98.0% by wt. potassium salt and residual lithium salt, or a mixture of 1.0-98.0% by wt. sodium salt and residual lithium salt, or a mixture of 1.0-0.55% by wt. potassium salt, 1.0-0.85% by wt. sodium salt and residual lithium salt.

In such case, 4-chlorine-2-methyl phenoxyacetic acid is mixed with its derivatives in a proportion of 1.0-98.0% by wt. MCPA and residual derivatives.

The medicament with immune-modulating, inflammation-inhibiting and antiblastomatous properties as well as antiviral activity contains 4-chlorine-2-methyl phenoxyacetic acid and/or its pharmacologically viable derivatives, for example, salts of alkali metals of the acid or mixtures of these salts or respectively mixtures of MCPA and derivatives thereof.

In such case, the derivative of 4-chlorine-2-methyl phenoxyacetic acid represents either a potassium salt or a sodium salt or a lithium salt of this acid.

Furthermore, the MCPA derivative represents either a mixture of potassium salt and sodium salt of the acid in a proportion of 1.0-98.0% by wt. potassium salt and residual sodium salt, or a mixture of 1.0-98.0% by wt. potassium salt and residual lithium salt, or a mixture of 1.0-98.0% by wt. sodium salt and residual lithium salt, or a mixture of 1.0-0.55% by wt. potassium salt, 1.0-0.85% by wt. sodium salt and residual lithium salt.

In such case, 4-chlorine-2-methyl phenoxyacetic acid is mixed with its derivatives in a proportion of 1.0-98.0% by wt. MCPA and residual derivatives.

4-chlorine-2-methyl phenoxyacetic acid is obtained by means of a chlorination of o-kresol, an additional reaction with chloracetic acid and a removal of the target product from the obtained mixture, for example by recrystallizing.

Salts of alkali metals are obtained in the following form:

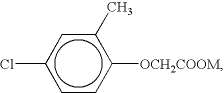

M being=K, Na, Li,
for example by means of the interaction of an aqueous solution of the acid either with potassium hydroxide or sodium hydroxide or lithium hydroxide and required mixtures by selecting specific constituent ingredients in a prescribed ratio.

Experiments carried out showed that 4-chlorine-2-methyl phenoxyacetic acid and its pharmacologically viable derivatives in the form of alkali metal salts as well as medicaments containing appropriate mixtures of these salts, which promote cell and humoral protective factors, have an inflammation-inhibiting and antiblastomatous effect, which is expressed for example in a therapeutic effect, ascertained by the inventor and called “disymmetrical”. This effect is defined in the temperature measured at various body points, for example 2-4° C. in 0.5-2 hours after the introduction of medicament (see M. W. Kutuschow, Cancer, healing is possible, Verlag Newa, St. Petersburg, 2003). The medicament has an antiviral activity.

This is confirmed by laboratory tests as well as patient examinations of the blood counts of the leucocyte and lymph systems of the patients prior and subsequent to the introduction of the preparation.

The preparation, obtained on the basis of the drug, may be taken orally as a powder or as tablets during or after a meal, its administration in a wide dose range, such as from 10 mg to 4 g, causing no allergic reactions and other side effects, and its effectiveness (in the treatment of appropriate illnesses) being not less than that of the prototype.

Test results of the effective mechanism of the drug according to this invention are listed in the following Examples 1-6, and the possible uses of the drug are confirmed by the following Examples 7-12.

### Example 1

The drug is introduced into test tubes of a trial group with cancer cells PC-3 (prostate carcinoma) in a buffer solution (10 ml) in a dilution of 10⁻⁵ mmol/l in the form of 4-chlorine-2-methyl phenoxyacetic acid, its potassium salts, sodium salts and lithium salts, mixtures of potassium salt and sodium salt, potassium salt and lithium salt, sodium salt and lithium salt, potassium salt, sodium salt and lithium salt (in equal ratio of the constituent ingredients) and mixtures of this acid and potassium salt, sodium salt and lithium salt (in the proportion 25% by wt. acid and residual salt).

Furthermore, the preparations Adriamycin, Reaferon and physiological saline solution are introduced into test tubes with the same cancer cell pool.

In such case, a control group with the same number of test tubes and the same components except cancer cells was formed, and fibroblasts instead of cancer cells were introduced into the test tubes of this group.

After thermostatically controlling the aforementioned test tubes in the course of 24 hours at a temperature of 37° C., the contents were analyzed.

The results obtained showed:

In the trial group with the medicament, the mitochondrial membranes were practically completely destroyed, the appearance for all of the compositions being virtually identical. In the test tubes with Adriamycin, a membrane swelling was observed, with Reaferon a membrane swelling with partial (approx. 60%) destruction was observed. The cells in the test tubes with physiological saline solution showed no change.

In the test tubes of the control group, the mitochondrial membranes are swollen, but their completeness is maintained.

The obtained data prove that the medicament according to this invention causes a destruction of the mitochondrial membranes of cancer cells, but does not destroy the structure of the normal cells. In other words, the drug develops in an intense manner the capacity to form ferments, which cause a cancer cell apoptosis.

### Example 2

Into four test tubes of a trial group with cancer cells MCF-7 (mammary gland adenocarcinoma) in a buffer solution (10 ml), the drug was introduced in a dilution of 10⁻⁵ mmol/l in the form of 4-chlorine-2-methyl phenoxyacetic acid and mixtures of this acid with potassium salts, sodium salts and lithium salts in two ratios: 1.) 25% by wt. acid, 25% by wt. potassium salt, 25% by wt. sodium salt and 25% by wt. lithium salt; 2.) 70% by wt. acid, 10% by wt. potassium salt, 10% by wt. sodium salt and 10% by wt. lithium salt; 3.) 10% by wt. acid, 10% by wt. potassium salt, 55% by wt. sodium salt and 25% by wt. lithium salt.

For the control group with the same cancer cell pool, a physiological saline solution is introduced into one test tube and Adriamycin and Reaferon are introduced into the remaining test tubes.

In the two test tube groups, titres of Cytochrome C are determined which total 1:14000.

After thermostatically controlling the mentioned test tubes in the course of 24 hours at a temperature 37° C., the contents were analyzed.

The results obtained showed:

In the test tubes of the trial group with the introduced drug, the titre of Cytochrome C in the test tube with MCPA amounts to 1:2000, and in the test tubes with the drug in the form of mixtures of this acid with appropriate salts the titre amounts to 1: (1950-2250).

In the test tubes with Adriamycin and Reaferon, the titre amounts to 1:12000 or respectively 1:9600. In the test tube with the physiological saline solution, no particular change in titre is observed.

The obtained results lead to the assumption that the drug promotes a release of an “aggressive” protein of Cytochrome C from the membranes of the cancer cell mitochondria, which causes apoptosis of said cells, triggering the destruction mechanism of the deoxyribonucleic acid through caspases (see Wilson, B. E., Mochon, E. A., Boxer, L. M., Induction of blc-2 expression by phosphorylated CREB proteins during B-cell activation and rescue from apoptosis, Mol. Cell. Biol., 1996, Vol. 16, P. 5546-5556).

### Example 3

Into a test tube (10 ml) with Transthyretin (protein) of the blood plasma (pH=7.0) in a concentration of 0.05 mmol/ml, four drops of 0.1% MCPA aqueous solution were introduced. The test tube was thermostatically controlled at a temperature of 37° C. After 15 minutes, the concentration of Transthyretin and the pH value of the blood plasma were measured. Transthyretin was not found, and the pH value was 6.0.

It thus follows that the drug works on the protein denaturation not as an acid, but as a preparation which alters the polymeric structure. This property causes the therapeutic effect in the treatment of malignant tumours.

### Example 4

Into a test tube with β-amyloid (protein) of the blood plasma (pH=7.4) with a concentration of 0.1 mmol/ml, three drops of 0.1% sodium salt aqueous solution (called O.F.1) are introduced. The test tube was thermostatically controlled at a temperature of 37° C. After 15 minutes, the protein concentration and the pH value of the blood plasma were measured. The protein concentration amounted to 0.03 mmol/ml, and the pH value amounted to 7.2.

It thus follows that the preparation O.F.1 prevents the denaturation of the amyloid precursor protein, and its conversion into β-amyloid. This property causes the therapeutic effect in the treatment of amyloidoses and cancer.

### Example 5

The blood of a sarcoma patient (10 ml) was centrifuged. A proportion of plasma was dried and detected in the polarisation microscope. Into the remaining proportion of plasma, an aqueous solution of lithium salt of MCPA acid was introduced, and the mixture was also detected in the polarization microscope.

Results: In the first composition, a light diffusion took place, and in the second composition a double refraction of rays took place. These data suggest that the drug corrects the protein development altered because of the cancer.

### Example 6

A filtrate formed from melanoma 16 (10 ml) was accommodated in a quartz vessel and detected in the polarisation microscope. Thereafter, sodium salt (O.F.1) was introduced into the filtrate, and a repeat detection was carried out in the polarisation microscope.

Results: On the filtrate image without the preparation, the light polarization is slight, and on the image with the preparation, a pronounced polarization of the ray, passing through the composition, was observed. The obtained data prove that the drug causes structural changes in the protein of cancer cells.

### Example 7

The reaction to drugs in the form of 4-chlorine-2-methyl phenoxyacetic acid and its potassium, sodium and lithium salts was tested on mice of the B-57 breed.

The trial group included 40 mice, and was split into 4 sub-groups of 10 mice each. The control group included 10 mice. In such case, the sub-groups of the trial group were kept separate from the control group. The mice of each sub-group were designated from 1 to 10 with a dye, in this case with the viride nitens.

The trial group received the drug during the course of 10 days: the 1st sub-group was given 4-chlorine-2-methyl phenoxyacetic acid, the 2nd sub-group was given the potassium salt of the acid, the 3rd sub-group was given the sodium salt of the acid (preparation O.F.1) and the 4th sub-group was given the lithium salt of the acid. In such case, mice Nos. 1-8 received the preparation as a drink (in an aqueous solution) and Nos. 9-10 received the preparation as injections into the peritoneum. In such case, in each sub-group the single dose for mice Nos. 1-2 was 2 mg, for Nos. 3-4 was 10 mg, for Nos. 5-6 was 15 mg, for Nos. 7-8 was 20 mg and for Nos. 9-10 was 5 mg per 1 ml of injection water. The mice of the control group received no preparation.

Results: Two weeks after the introduction of the drug, all of the mice of the trial group and control group were still alive. No differences in the behavior of the trial group and control group were observed. The mice with even numbers in all of the sub-groups were killed on the 21st day.

Traces of the drug were ascertained in the kidneys of No. 8 in all of the sub-groups of the trial group. During a histological examination, no differences in the effect of 4-chlorine-2-methyl phenoxyacetic acid and its salts on the organism were ascertained.

It can thus be assumed, with respect to weight ratios, that the single dose of 4.0 g is viable for the human organism. More accurate data, however, can only be specified after appropriate investigations.

### Example 8

The mice of the B-57 breed of the trial group and control group (60 mice each) were inoculated with melanoma-16. The trial group and control group were split into 3 sub-groups each with 20 mice. The 1st-3rd sub-groups of the trial group received the drug as a drink in a single dose of 5 mg in aqueous solutions of 4-chlorine-2-methyl phenoxyacetic acid, its sodium salt (preparation O.F.1) and the mixture of MCPA as well as potassium salt and sodium salt in a ratio of 25:25:50% by wt. The first and second sub-groups of the control group received aqueous solutions of Adriamycin and Reaferon as a drink in a single dose of 5 mg. The third sub-group received no preparations.

Results: On the 36th day of the test, 22 out of 30 mice in the trial group were still alive. 4 mice (2 from the first sub-group, one mouse each from the second and third sub-groups) died on the 23rd day, 3 mice (one from the second sub-group and 2 from the third sub-group) died on the 26^{th} day, and 1 mouse (from the second sub-group) died on the 30th day of the test. 15 surviving mice (3 from the first sub-group, 8 from the second sub-group and 4 from the third sub-group) had blastomas with a size of approximately 0.3×0.2 mm. In the case of 7 mice (3 from the first sub-group, one from the second sub-group and 3 from the third sub-group), no blastomas were ascertained. The surviving mice were killed on the 36th day of the test. During a dissection, isolated pulmonal metastases were ascertained in the case of 8 mice (2 from the first sub-group, 4 from the second sub-group and 2 from the third sub-group). The internal organs remained without visible changes.

In the first and second sub-groups of the control group, 2 mice from the first sub-group and 4 mice from the second sub-group survived until the 36th day of the test. Blastomas had an average size of 2.0×1.0 cm. During the dissection, lung and liver damage were ascertained.

All of the mice from the third sub-group of the control group died on the 12th, 15th, 20th, 25th and 26th days. In the investigation, bleeding back blastomas with an average size of 2.5×2.0 cm were ascertained. A histological investigation showed a total lung lysis.

Conclusions: The drug has a pronounced antiblastomatous effect and causes no side effects.

### Example 9

An ill patient K. of 69 years had, as disorders, a painful urination as well as a urinary blockage and blood in the urine. An ultrasound test on 8 Aug. 2003 showed the prostate with a size of approximately 90 cm³ and a tumour with indefinite contours and a size of 35×48 mm². A PSA test on 8 Aug. 2003 revealed 27.9 mg/ml. A puncture biopsy showed an adenocarcinoma of the prostate gland.

The diagnosis revealed an adenocarcinoma of the prostate gland. The patient declined an operation and chemotherapy. The patient was treated with the drug. On 9 Aug. 2003, the treatment with 4-chlorine-2-methyl phenoxyacetic acid in a single dose of 300 g twice daily during meals was started. The treatment lasted 10 days. In this period of time, the pains during urination became less, and the urine colour was correct. An ultrasound test showed that the prostate had become smaller by 30%. The tumour had a size of 12×24 mm² and sharp contours.

Thereafter, the therapy was continued for the course of 2 weeks with a mixture of 4-chlorine-2-methyl phenoxyacetic acid, potassium salt and sodium salt in a ratio of 50:10:40 per 400 g twice daily, as well as with a daily enema prior to going to sleep with 2.0 g sodium salt of MCPA acid, dissolved in 50 ml water. During the subsequent investigation, the pathological symptoms had completely disappeared, according to the statement of the patient (no painful urination, no urinary blockage, inter alia).

A control examination with ultrasound showed a size of the prostate of approximately ≈42 cm³ and a tumour of 6×6 mm². A PSA test on 8 Oct. 2003 revealed 4.1 mg/ml.

The patient was recommended to take the 4-chlorine-2-methyl phenoxyacetic acid preparation in a dose of 200 mg twice daily during meals for the course of a month, as well as to use daily enemas prior to going sleep, each with 2.0 g sodium salt and potassium salt of the acid (in the ratio 50:50% by wt.), dissolved in 50 ml water.

The blood counts of the laboratory test are listed in the following Table:

| | | |
|---|---|---|
| Before | after | after |
| treatment | 2 weeks | 2 months |

General blood test

| | | | |
|---|---|---|---|
| Haemoglobin, g/l | 100 | 134 | 145 |
| Erythrocytes, 1 × 10¹²/l | 3.5 | 4.3 | 4.7 |
| Haemoglobin index | 0.75 | 0.85 | 0.96 |
| Leucocytes, 1 × 10⁹/l | 3.0 | 4.7 | 6.0 |
| Thrombocytes, absolute (Tsd.) | 156 | 323 | 354 |
| Eosinophiles, % | 3.0 | 3.0 | 2.0 |

Neutrophiles:

| | | | |
|---|---|---|---|
| Standard bar core, % | 6.0 | 6.0 | 6.0 |
| Segment core, % | 69 | 71 | 72 |
| Lymphocytes, % | 21 | 22 | 26 |
| Monocytes, % | 4.5 | 6.0 | 5.8 |
| BKS mm/h | 30 | 14 | 12 |

Biochemical blood test

| | | | |
|---|---|---|---|
| Glucose mmol/l | 3.6 | 4.9 | 4.0 |
| Urea mg/dl | 43.1 | 41.1 | 38.7 |
| Uric acid mg/dl | 5.2 | 6.3 | 6.4 |
| Albumin g/l | 39.4 | 49.6 | 50.1 |
| Protein | 81.1 | 82.9 | 88.9 |
| Creatine mg/dl | 0.43 | 0.39 | 0.35 |
| Aspart aminotransferasis | 34.0 | 28.0 | 31.0 |
| units/l | | | |
| Alanine aminotransferasis | 77.0 | 71.4 | 55.9 |
| units/l | | | |
| Gammaglutamyl transferasis | 129.0 | 82.0 | 68.6 |
| units/l | | | |
| Lactate dehydrogenasis | 478.0 | 135.0 | 96.8 |
| units/l | | | |

Test results cell and humoral immunity:

| | | | |
|---|---|---|---|
| Immune globulin A, g/l | 2.4 | 2.31 | 2.59 |
| Immune globulin M, g/l | 1.9 | 1.52 | 1.65 |
| Immune globulin G, g/l | 11.0 | 10.5 | 10.6 |
| T-lymphocytes, % | 54.0 | 62.0 | 68.0 |
| B-lymphocytes, % | 14.0 | 21.0 | 32.0 |
| Latex phagocytoses, % | 54 | 55 | 58 |
| TNF | 15.6 | 23.0 | 32 |
| TH (Helper cell), % | 24.0 | 31.0 | 25.0 |

### Example 10

An ill lady A. of 43 years underwent an extirpation of the womb and the uterus because of a carcinoma of the right ovary in the year 2000. After the operation, two years of chemotherapy treatments followed. At the end of July 2002, rectal and tenesmus pains began. A computer tomogram on 18 Aug. 2002 showed a tumour with a size of 17.0×15.9 cm² between the rectum and the urinary bladder, which was growing through the walls thereof. A tumour recidive was diagnosed. In view of the ineffective chemotherapy, a treatment with the preparation was carried out: in the course of 14 days, 400 mg of the potassium salt of 4-chlorine-2-methyl phenoxyacetic acid were taken three times per day, and daily enemas with 2.0 g mixture of 4-chlorine-2-methyl phenoxyacetic acid and the potassium salt of this acid (in the ratio 90:10% by wt.) were used; thereafter, in the course of 21 days, a mixture of respectively 500 mg potassium salt and sodium salt of the acid (in the ratio 25:75% by wt.) was administered twice a day, and daily enemas with 2.0 g mixture of 4-chlorine-2-methyl phenoxyacetic acid, the potassium salt and the sodium salt of this acid (in the ratio 25:25:50% by wt.) were used.

On the 3rd day of the treatment, the pains became weaker, and the urge to urinate and defecate became less frequent. An ultrasound examination after 2 weeks revealed a tumour size of 7.4×5.7 cm²; after a further 2 weeks it was 1.2×2.3 cm², and there was a sharp boundary between the tumour and the rectum wall and urinary bladder wall. A control examination with ultrasound after 2 months showed a round, sharply defined structure with a size of 0.6×0.5 cm², into which a thick, pulsating artery penetrated. An additional examination showed an adequate state, while an ultrasound examination on 23 Feb. 2004 showed a sharply defined structure with a size of 0.2×0.1 cm² without a visible circulation of blood. No exudate was found in the small pelvis.

The blood counts of the laboratory test are listed in the following Table:

| | | |
|---|---|---|
| Before | after | after |
| treatment | 2 weeks | 2 months |

General blood test

| | | | |
|---|---|---|---|
| Haemoglobin, g/l | 97 | 123 | 149 |
| Erythrocytes, 1 × 10¹²/l | 2.5 | 3.3 | 4.7 |
| Haemoglobin index | 0.75 | 1.0 | 1.0 |
| Leucocytes, 1 × 10⁹/l | 3.0 | 4.7 | 5.5 |
| Thrombocytes, absolute (Tsd.) | 164 | 223 | 259 |
| Eosinophiles, % | 1.0 | 2.0 | 1.0 |

Neutrophiles:

| | | | |
|---|---|---|---|
| Standard bar core, % | 5.7 | 5.9 | 6.0 |
| Segment core, % | 71 | 69 | 68 |
| Lymphocytes, % | 22 | 24 | 25 |
| Monocytes, % | 5.5 | 5.0 | 5.9 |
| BKS mm/h | 33 | 16 | 10 |

Biochemical blood test

| | | | |
|---|---|---|---|
| Glucose mmol/l | 4.6 | 5.9 | 5.0 |
| Urea mg/dl | 42.5 | 44.0 | 28.9 |
| Uric acid mg/dl | 4.9 | 6.8 | 5.4 |
| Albumin g/l | 40.4 | 47.8 | 50.4 |
| Protein g/l | 61.9 | 71.6 | 81.8 |
| Creatine mg/dl | 0.42 | 0.35 | 0.34 |
| Aspart aminotransferasis | 33.0 | 31.0 | 28.0 |
| units/l | | | |
| Alanine aminotransferasis | 77.0 | 71.4 | 55.9 |
| units/l | | | |
| Gammaglutamyl transferasis | 129.0 | 82.0 | 68.6 |
| units/l | | | |
| Lactate dehydrogenasis | 543.5 | 335.0 | 126.8 |
| units/l | | | |

Test results of the cell and humoral immunity:

| | | | |
|---|---|---|---|
| Immune globulin A, g/l | 2.1 | 2.30 | 2.29 |
| Immune globulin M, g/l | 1.6 | 1.5 | 1.75 |
| Immune globulin G, g/l | 10.9 | 11.0 | 10.8 |
| T-lymphocytes, % | 56.0 | 61.0 | 65.0 |
| B-lymphocytes, % | 12.0 | 19 | 31.0 |
| Latex phagocytoses, % | 51 | 55 | 56 |
| TNF | 14.9 | 21.0 | 34.5 |
| TH (Helper cell), % | 23.0 | 29.0 | 26.0 |

### Example 11

NHL (non-Hodgkin's lymphoma) was diagnosed in a sick woman U. of 35 years. A course of chemical preparations and radiation treatments followed from 1994. The condition deteriorated despite massive inflammation-inhibiting therapy and hormone therapy. The lymph nodes were greatly enlarged (the largest had a diameter of 18-20 cm); dyspnoea and debility occurred.

A treatment with 4-chlorine-2-methyl phenoxyacetic acid and its derivatives followed. From 12 Feb. 2003, the sick woman took the preparation, in a quantity of 400 mg each twice a day per os during mealtimes, which preparation contained a mixture of 4-chlorine-2-methyl phenoxyacetic acid and the sodium salt of this acid (in a ratio of 30:70% by wt.). In two weeks, the condition improved. The dyspnoea disappeared. The appetite returned. The visible nymph nodes became smaller by the factor of 1.5-2.

After three weeks, the preparation was prescribed, in a quantity of 300 mg each twice a day for taking during mealtimes, which preparation contained a mixture of sodium salt and lithium salt of 4-chlorine-2-methyl phenoxyacetic acid (in the ratio 75:25% by wt.).

After 1.5 months from the start of the treatment, the lymph nodes had completely disappeared (confirmed by ultrasound examination). The condition was adequate, and no disorders occurred. A control examination after a year revealed no disorders and no recidives.

The blood counts of the laboratory test are listed in the following Table:

| | | |
|---|---|---|
| Before | after | after |
| treatment | 2 weeks | 2 months |

General blood test

| | | | |
|---|---|---|---|
| Haemoglobin, g/l | 130 | 134 | 145 |
| Erythrocytes, 1 × 10¹²/l | 3.5 | 4.3 | 4.7 |
| Haemoglobin index | 1.05 | 1.05 | 1.3 |
| Leucocytes, 1 × 10⁹/l | 3.0 | 4.7 | 5.5 |
| Thrombocytes, absolute (Tsd.) | 140 | 325 | 410 |
| Eosinophiles, % | 3.0 | 3.0 | 1.0 |

Neutrophiles:

| | | | |
|---|---|---|---|
| Standard bar core, % | 5.1 | 5.0 | 4.5 |
| Segment core, % | 62 | 56 | 54 |
| Lymphocytes, % | 24 | 21 | 25 |
| Monocytes, % | 0.5 | 0.4 | 5.0 |
| BKS mm/h | 43 | 32 | 9 |

Biochemical blood test

| | | | |
|---|---|---|---|
| Glucose mmol/l | 3.1 | 4.7 | 4.9 |
| Urea mg/dl | 44.1 | 44.1 | 38.0 |
| Uric acid mg/dl | 6.2 | 8.3 | 7.4 |
| Albumin g/l | 37.4 | 49.6 | 50.1 |
| Protein g/l | 80.1 | 82.5 | 82.9 |
| Kreatinin mg/dl | 0.45 | 0.31 | 0.35 |
| Aspart aminotransferasis | 36.0 | 29.0 | 30.0 |
| units/l | | | |
| Alanine aminotransferasis | 75.0 | 65.7 | 46.9 |
| units/l | | | |
| Gammaglutamyl transferasis | 107.0 | 98.0 | 45.6 |
| units/l | | | |
| Lactate dehydrogenasis | 459.0 | 230.0 | 217.4 |
| units/l | | | |

Test results of the cell and humoral immunity:

| | | | |
|---|---|---|---|
| Immune globulin A, g/l | 2.05 | 2.32 | 2.53 |
| Immune globulin M, g/l | 1.9 | 1.57 | 1.56 |
| Immune globulin G, g/l | 11.0 | 11.5 | 11.6 |
| T-lymphocytes, % | 54.0 | 65.0 | 69.0 |
| B-lymphocytes, % | 15.0 | 24.0 | 42.0 |
| Latex phagocytoses, % | 43 | 80 | 67 |
| TNF | 15.6 | 23.0 | 32.3 |
| TH (Helper cell), % | 26.0 | 30.0 | 28.0 |
| TS (Suppressor cell), % | 23.0 | 28.0 | 31.0 |

### Example 12

A sick man N. of 27 years was diagnosed with hepatitis C, hepatargy and ascites. The sick man was infected in 1989 by medicinal tools or a blood transfusion after a stomach operation because of stomach damage with the virus of hepatitis C. The first pathological symptoms appeared 9 years after the infection, namely pains in the right lower ribs, jaundice and poor biochemical blood counts. After treatment in hospital, the condition improved. Recidively chronic hepatitis occurred after influenza in the year 2001. Furthermore, ascites and leg oedema, sub-febrile temperature and jaundice, occurred. In November 2002, the sick man was offered a liver transplant, which he declined.

In January 2003, treatment with the drug was started. In the course of 2 weeks, the patient took 4-chlorine-2-methyl phenoxyacetic acid in a quantity of 250 mg each twice a day, and thereafter the mixture of the acid and the potassium salts, sodium salts and lithium salts of this acid in equal ratios in a quantity of 400 mg each three times a day during mealtimes. In the course of a month, the blood transaminasis reduced considerably. The jaundiced look was reduced. Pruritus, pains in the right lower ribs and excessive perspiration had disappeared. After a month, the single dose of the preparation was doubled. After two months, the condition was satisfactory. No leg oedema and no ascites occurred. The skin was yellowish, and the eyeball membranes were clear.

The patient was recommended to take the preparation in the form of a mixture of potassium salt and lithium salt of 4-chlorine-2-methyl phenoxyacetic acid in equal ratio, each 250 mg, once to twice daily for a month.

The blood counts of the laboratory test are listed in the following Table:

| | | |
|---|---|---|
| Before | after | after |
| treatment | 2 weeks | 2 months |

General blood test

| | | | |
|---|---|---|---|
| Haemoglobin, g/l | 100 | 123 | 149 |
| Erythrocytes, 1 × 10¹²/l | 4.5 | 4.3 | 5.0 |
| Haemoglobin index | 0.85 | 1.0 | 1.0 |
| Leucocytes, 1 × 10⁹/l | 8.0 | 4.7 | 6.1 |
| Thrombocytes, absolute (Tsd.) | 104 | 127 | 224 |
| Eosinophiles, % | 3.0 | 4.0 | 1.0 |

Neutrophiles:

| | | | |
|---|---|---|---|
| Standard bar core, % | 4.6 | 4.9 | 5.0 |
| Segment core, % | 67 | 68 | 65 |
| Lymphocytes, % | 20 | 23 | 23 |
| Monocytes, % | 4.5 | 4.0 | 4.9 |
| BKS mm/h | 43 | 24 | 12 |

Biochemical blood test

| | | | |
|---|---|---|---|
| Sodium mmol/l | 139 | 143 | 143 |
| Potassium mmol/l | 3.0 | 4.2 | 4.5 |
| Glucose mmol/l | 4.6 | 5.9 | 5.0 |
| Urea mg/dl | 42.5 | 44.0 | 28.9 |
| Uric acid mg/dl | 4.9 | 6.8 | 5.4 |
| Bilirubin mg/dl | 2.9 | 1.7 | 0.8 |
| Albumin g/dl | 2.0 | 4.7 | 5.4 |
| Protein g/dl | 6.1 | 7.1 | 8.4 |
| Creatine mg/dl | 0.42 | 0.35 | 0.34 |
| Aspart aminotransferasis | 73.0 | 51.0 | 28.0 |
| units/l | | | |
| Alanine aminotransferasis | 77.0 | 71.4 | 43.9 |
| units/l | | | |
| Gammaglutamyl transferasis | 129.0 | 82.0 | 68.6 |
| units/l | | | |
| Lactate dehydrogenasis | 543.5 | 335.0 | 247.0 |
| units/l | | | |

1. A drug having immune-modulating, inflammation-inhibiting and antiblastomatous properties as well as an antiviral activity, the drug including at least one of 4-chlorine-2-methyl phenoxyacetic acid with a structural formula: 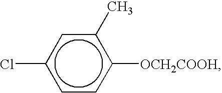
and pharmacologically viable derivatives of the acid, including one of salts of alkali metals of the acid and mixtures of the salts or respectively mixtures of 4-chlorine-2-methyl phenoxyacetic acid and the derivatives.

2. The drug according to claim 1, wherein the derivative of 4-chlorine-2-methyl phenoxyacetic acid is a potassium salt of the acid.

3. The drug according to claim 1, wherein the derivative of 4-chlorine-2-methyl phenoxyacetic acid is a sodium salt of the acid.

4. The drug according to claim 1, wherein the derivative of 4-chlorine-2-methyl phenoxyacetic acid is a lithium salt of the acid.

5. The drug according to claim 1, wherein the derivative of 4-chlorine-2-methyl phenoxyacetic acid is a mixture of 1.0-98.0% by wt. potassium salt and residual sodium salt.

6. The drug according to claim 1, wherein the derivative of 4-chlorine-2-methyl phenoxyacetic acid is a mixture of 1.0-98.0% by wt. potassium salt and residual lithium salt.

7. The drug according to claim 1, wherein the derivative of 4-chlorine-2-methyl phenoxyacetic acid is a mixture of 1.0-98.0% by wt. sodium salt and residual lithium salt.

8. The drug according to claim 1, wherein the derivative of 4-chlorine-2-methyl phenoxyacetic acid is a mixture of 1.0-55.0% by wt. potassium salt, 1.0-85.0% by wt. sodium salt and residual lithium salt.

9. The drug according to claim 1, wherein the mixture of 4-chlorine-2-methyl phenoxyacetic acid and derivatives is accomplished in a proportion of 1.0-98.0% by wt. 4-chlorine-2-methyl phenoxyacetic acid and residual derivatives.
